(19) United States
(12) Patent Application Publication
(10) Pub. No.: US 2008/0182812 A1
(43) Pub. Date: Jul. 31, 2008

(54) DISEASE MODEL ANIMALS OF METABOLIC SYNDROME AND A METHOD OF SCREENING PREVENTIVE AND THERAPEUTIC AGENTS FOR METABOLIC SYNDROME USING THE SAME

(75) Inventors: Junji Yodoi, Kyoto (JP); Hiroshi Masutani, Kyoto (JP); Shinichi Oka, Kyoto (JP); Hajime Nakamura, Osaka (JP)

Correspondence Address: BROWN & MICHAELS, PC;400 M & T BANK BUILDING, 118 NORTH TIOGA ST, ITHACA, NY, 14850, US

(73) Assignee: REDOX BIOSCIENCE INC., Kyoto (JP)

(21) Appl. No.: 12/026,718

(22) Filed: Feb. 6, 2008

Related U.S. Application Data
(62) Division of application No. 11/553,125, filed on Oct. 26, 2006.

(51) Int. Cl. A61K 31/711

(52) U.S. Cl. 514/44

(57) ABSTRACT

The object of the invention is to provide animal model of disorder and method of screening an agent for preventing or treating Metabolic Syndrome by using the animal model, wherein said animal model is used as experimental material which is essential to detailed analysis of component and pathologic condition of Metabolic Syndrome and to development of the method for treating and the agent for preventing and treating the Metabolic Syndrome.

The above object is achieved by the non-human mammal model of disorders, whose TBP-2 gene is functionally deficient on chromosome, wherein the disorders are caused by impaired fatty acid utilization, and a method of screening an agent for preventing or treating Metabolic syndrome comprising; administering a test article to the non-human mammal whose TBP-2 gene is functionally deficient on chromosome.

### REFERENCE TO RELATED APPLICATIONS

This is a divisional patent application of copending application Ser. No. 11/553,125, filed Oct. 26, 2006, entitled “Animal model of disorders caused by impaired fatty acid utilization and method of screening an agent preventing or treating Metabolic Syndrome by using said animal model”. The aforementioned application is hereby incorporated herein by reference.

### TECHNICAL FIELD

The present invention relates to animal model of disorders caused by impaired fatty acid utilization and method of screening an agent for preventing or treating Metabolic Syndrome by using said animal model.

In detail, the present invention relates to animal model of disorders caused by impaired fatty acid utilization wherein thioredoxin binding protein-2 (TBP-2) gene is functionally deficient on a chromosome in the animal model, and method of screening an agent for preventing or treating Metabolic Syndrome by using said animal model.

### DESCRIPTION OF THE RELATED ART

Thioredoxin (hereinafter referred to as TRX) is reported as a multi-functional peptide with a molecular weight of 12 kDa having a redox activity derived from the disulfide/dithiol exchange reaction in its active amino acid sequence (-Cys-Gly-Pro-Cys-) (See Redox regulation of cellular activation Ann. Rev. Immunol 1997; 15:351-369). Since TRX exhibits radical scavenging ability and anti-oxidant ability, it works as functional protein controlling intracellular or extracellular redox environment. Because TRX controls activity of factors involved in redox reactions such as NF-κB or AP-1 (activator protein-1) and controls activities of p38 mitogen activating protein kinase (p38MAPK) and of apoptosis signal regulating kinase-1 (ASK-1), it is thought to be greatly involved in cell proliferation and apotosis signaling. (“AP-1 transcriptional activity is regulated by a direct association between thioredoxin and Ref-1” PNAS. 1997; 94:3633-3638).

TRX Binding Protein-2 (hereinafter referred to as TBP-2) has recently been identified as the protein binding with TRX by yeast two-hybrid method. (Nishiyama, A. et al., J. Biol. Chem., 274(31):21645-50, 1999)

TBP-2 is identical with vitamin D3 upregulated protein 1 (VDUP-1) which is induced by vitamin D3. TBP-2 is thought as an endogenous inhibitor of TRX, because it selectively binds with reduced TRX and suppresses reducing-activity of the TRX. (Kiich Hirota et al., protein/nucleic acid/enzyme 44(15): 2414-2419, 1999)

It has been reported that TBP-2 not only suppresses reducing-activity of TRX but also has broad biological activities as described later. For example, it is known that expression of TBP-2 is significantly reduced in many kinds of cancer-cells and that cell proliferation is suppressed by excess-expression of TBP-2. This allows us to consider TBP-2 as a factor suppressing cell-proliferation and cancer. It is reported that cell proliferation suppression mechanism of TBP-2 includes suppressions of phosphorylation of Rb protein and hyperfunction of transcriptional repression complex. Said suppressions of phosphorylation of Rb protein mean suppressions occurring when expression of cell proliferation-suppressor p 16 is increased (Nishinaka, Y. et al.: Cancer Res., 64(4): 1287-1292, 2004). Said transcriptional repression complex is the complex obtained by binding with promyeol-cytic leukemia zinc-finger (PLZF) and histon deacetylase 1 (HDAC1) (Han, S. H. et al.: Oncogene, 22(26):4035-4046). It is thought that functional expression of TBP-2 in nuclear is related to transfer of TBP-2 in nuclear in importin system (Nishinaka, Y. et al.:J. Biol. Chem., 279(36):37559-37565, 2004).

### DISCLOSURES OF THE INVENTION

### Problem to be Solved by the Invention

The number of patients suffering from a group of disorders called Metabolic Syndrome which is developed with hypertension, hyperlipidemia, diabetes or obesity is significantly increased, while average life span is increased. (For example, it is reported that the number of people with diabetes reaches about 7.4 million, and the number of the people including the people-to-be reaches about 16.2 million which is over 10% of Japanese population in these days in Japan)

Metabolic Syndrome is known as a disorder caused or developed by daily life style such as eating habit, insufficient exercise, insufficient relaxation, smoking or drinking as well as genetic factor or external environmental factor including pathogen, harmful substance, stressor or the like. Metabolic Syndrome is a narrowly defined lifestyle-related disease.

Metabolic Syndrome brings pathologic condition typified by hypertension, hyperlipidemia, diabetes or obesity. Each of those causes atherosclerosis, and it is not uncommon that more than two of those are complicated in an individual. In such a case, risk for atherosclerosis is significantly increased.

Recently it has been imperative that pathologic condition of Metabolic Syndrome are completely analyzed, that pathogenic mechanism of it is fully revealed and that agents for preventing and treating it is more developed. To accomplish those tasks we face, animal model of phenotypic disorder is necessary, which is with almost the same pathologic condition as Metabolic Syndrome. Thus because such an animal model can be used as targets in experiments, it will greatly help us.

Development of the animal model of phenotypic disorder of Metabolic Syndrome is eagerly hoped, and some transgenic animals and knockout animals with pathologic condition of Metabolic Syndrome have already been discovered and used. However, those animals can not satisfy our needs, because there is still difference on a genetic level and in pathologic condition between the mouse and human patients. Further, because the Metabolic Syndrome results in complexly interrelated multiple-pathologic condition as mentioned above, the development of animal model of phenotypic disorder remains undone.

In view of the above-circumstance, an object of the invention is to provide animal model of disorder and method of screening an agent for preventing or treating Metabolic Syndrome by using the animal model, wherein said animal model is used as experimental material which is essential to detailed analysis of component and pathologic condition of Metabolic Syndrome and to development of the method for treating it and the agent for preventing and treating the Metabolic Syndrome.

### The Means of Solving the Problems

The present inventors have concluded that animal model whose thioredoxin binding protein-2 (TBP-2) gene is functionally deficient on its chromosome (TBP-2 knockout mouse “TBP-2 knockout mouse (−/−)”) is used as an animal model of disorders caused by impaired fatty acid utilization or as an animal model having pathologic condition of hyperlipidemia. Further, it is has been concluded that such an animal model can be used for screening an agent for preventing or treating Metabolic Syndrome (especially disorders caused by impaired fatty acid utilization) so that the present invention has been completed.

Thus, the present invention provides a non-human mammal comprising a TBP-2 gene, wherein the gene is functionally deficient on a chromosome.

The present invention provides a non-human mammal model comprising a TBP-2 gene, wherein the gene is functionally deficient on a chromosome.

In an embodiment of the present invention, the model is a model of at least one disorder caused by impaired fatty acid utilization.

In an embodiment of the present invention, the non-human mammal model is used for screening an agent for preventing or treating at least one metabolic syndrome, disorder or pathologic condition.

In an embodiment of the present invention, the impaired fatty acid utilization is caused by a defect of TCA cycle.

In an embodiment of the present invention, the model is a model of a pathologic condition-of hyperlipidemia.

In another embodiment of the present invention, the non-human mammal is used for screening an agent for preventing or treating at least one metabolic syndrome, disorder or pathologic condition.

In yet another embodiment of the present invention, the non-human mammal is used for screening an agent for preventing or treating diabetes.

In another embodiment of the present invention, the model is a model of a disorder selected from the group consisting of Reye Syndrome, Reye-like Syndrome, fatty acid oxidation defect and acute fatty liver of pregnancy.

In another embodiment of the present invention, the model is a model of a pathologic condition selected from the group consisting of dysregulation of lipid metabolism, dysregulation of glucose metabolism and coagulation dysfunction.

In an embodiment of the present invention, the non-human mammal exhibits hemorrhage under a fasting condition.

In an embodiment of the present invention, the non-human mammal is a rodent. In one embodiment of the present invention, the rodent is a mouse.

The present invention also provides a method of screening an agent for preventing or treating at least one metabolic syndrome, disorder or pathologic condition comprising the step of administering a test article to a non-human mammal comprising a TBP-2 gene, wherein the gene is functionally deficient on a chromosome.

In one embodiment of the method of the present invention, the metabolic syndrome, disorder or pathologic condition is a disorder caused by impaired fatty acid utilization.

In another embodiment of the method of the present invention, the metabolic syndrome, disorder or pathologic condition is a metabolic syndrome.

In another embodiment of the method of the present invention, the metabolic syndrome, disorder or pathologic condition is hyperlipidemia.

In another embodiment of the method of the present invention, the metabolic syndrome, disorder or pathologic condition is diabetes.

In another embodiment of the method of the present invention, the metabolic syndrome, disorder or pathologic condition is selected from a group consisting of Reye Syndrome, Reye-like Syndrome, fatty acid oxidation defect and acute fatty liver of pregnancy.

In another embodiment of the method of the present invention, the metabolic syndrome, disorder or pathologic condition is a pathologic condition selected from a group consisting of dysregulation of lipid metabolism, dysregulation of glucose metabolism and coagulation dysfunction.

In an embodiment of the method of the present invention, the non-human mammal is a rodent.

In an embodiment of the method of the present invention, the non-human mammal is a mouse.

In an embodiment of the method of the present invention, the method further comprises the steps of administering the test article to a wild non-human mammal, comparing conditions between the two non-human mammals, and evaluating the conditions.

### EFFECTS OF THE INVENTION

The TBP-2 knockout non-human mammal according to the present invention is effectively used as an animal model of disorders or an animal with pathologic conditions, wherein the disorders are caused by impaired fatty acid utilization or wherein the pathologic conditions are of hyperlipidemia.

In addition, such a non-human mammal is useful when analyzing pathologic condition of Metabolic Syndrome (especially developed linking to impaired fatty acid utilization, hyperlipidemia or the like) and interpreting pathogenic mechanism of Metabolic Syndrome, and the non-human mammal is helpful to analyze and interpret those on an individual level. It is also used as experimental material for screening an agent for preventing or treating Metabolic Syndrome. Further, because the non-human mammal exhibits almost the same pathologic condition as the pathologic condition of disorder selected from Reye Syndrome, Reye-like Syndrome, Mitochondrial fatty acid β oxidation defect and acute fatty liver of pregnancy, it is used as the non-human mammal model of such disorders.

The method of screening according to the present invention is the significantly beneficial, because the method is useful for screening an agent for preventing or treating disorders caused by impaired fatty acid utilization, hyperlipidemia, diabetes, Metabolic Syndrome (especially developed linking to impaired fatty acid utilization, hyperlipidemia or the like) and disorder groups typified by Reye Syndrome.

### BRIEF DESCRIPTION OF THE DRAWINGS

The file of this patent contains at least one drawing executed in color. Copies of this patent with color drawing(s) will be provided by the Patent and Trademark office upon request and payment of necessary fee.

FIG. 1A shows a structure of the targeting vector in generating TBP-2^{-/-} mice.

FIG. 1B shows southern blot analysis using TBP-2^{-/-} mice.

FIG. 1C shows Northern blot analysis using TBP-2^{-/-} mice.

FIG. 1D shows growth curve of body weights comparing between TBP-2^{-/-} mice and wild type mice.

FIG. 2 shows survival rate (%) in TBP-2^{-/-} mice (homozygous mice), in TBP-2^{+/-} mice (heterozygous mice) and wild type mice (+/+) under a fasted state.

FIG. 3 shows photographs of liver collected form the fasted wild type mice, the fasted TBP-2^{-/-} mice and the fed TBP-2^{-/-} mice.

FIG. 4 shows photographs of gastrointestinal region collected form the fasted wild type mice, the fasted TBP-2^{-/-} mice and the fed TBP-2^{-/-} mice.

FIG. 5 shows TBP-2 expression in liver, heart and lung.

FIG. 6A shows red blood cell count at the indicated time points during fasting.

FIG. 6B shows prothrombin time (PT) at the indicated time points during fasting.

FIG. 6C shows APTT at the indicated time points during fasting.

FIG. 6D shows anti-thrombin III activity at the indicated time points during fasting.

FIG. 6E shows fibrinogen concentration at the indicated time points during fasting.

FIG. 6F shows platelet count at the indicated time points during fasting.

FIG. 7A shows microscopic view of H & E for histological studies.

FIG. 7B shows microscopic view of Oil Red O-stained for histological studies.

FIG. 8C shows serum levels of AST (aspartate aminotransferase) at the indicated time points during fasting.

FIG. 8D shows serum levels of ALT (alanine aminotransferase) at the indicated time points during fasting.

FIG. 8E shows serum levels of LDH (lactate dehydrogenase) at the indicated time points during fasting.

FIG. 8F shows serum levels of BUN at the indicated time points during fasting.

FIG. 8G shows serum levels of potassium at the indicated time points during fasting.

FIG. 8H shows serum levels of sodium at the indicated time points during fasting.

FIG. 9A shows survival rate of TBP-2^{-/-} mice comparing between the two administered glucose and oleic acid in drinking water during fasting.

FIG. 9B shows each concentration of AST, ALT, LDH, BUN, Na and K in serum in fasted TBP-2^{-/-} mice and in wild type mice.

FIG. 10C shows serum levels of glucose in TBP-2^{-/-} mice under both feeding and fasting states.

FIG. 10D shows serum levels of insulin in TBP-2^{-/-} mice under both feeding and fasting states.

FIG. 10E shows serum levels of free fatty acids in TBP-2^{-/-} mice under both feeding and fasting states.

FIG. 11F shows serum levels of triglyceride in TBP-2^{-/-} mice under both feeding and fasting states.

FIG. 11G shows serum levels of total cholesterol in TBP-2^{-/-} mice under both feeding and fasting states.

FIG. 11H shows serum levels of phospholipids in TBP-2^{-/-} mice under both feeding and fasting states.

FIG. 12A shows concentration of ketone bodies in TBP-2^{-/-} mice under both feeding and fasting states.

FIG. 12B shows concentration of pyruvate in TBP-2^{-/-} mice under both feeding and fasting states.

FIG. 12C shows concentration of lactate in TBP-2^{-/-} mice under both feeding and fasting states.

FIG. 13A shows that TBP-2 has an important role in fatty acid utilization through acetyl-CoA consumption through augmentation of the Kerbs cycle.

FIG. 13B shows that disruption of TBP-2 reduces acetyl-CoA consumption, which serially leads to dysregulation of lipid and glucose metabolism, hepatic and renal failure, and coagulation dysfunction.

### DESCRIPTION OF THE PREFERRED EMBODIMENTS

This time, the present inventors have found that fatty acid utilization is impaired when TBP-2 is disrupted. They have also found that the TBP-2 is importantly involved in preventing from hemorrhage under fasting condition.

The present invention is to provide a non-human mammal whose TBP-2 gene is functionally deficient on chromosome for using as a non-human mammal model of disorders caused by impaired fatty acid utilization and for using as a non-human mammal model with pathologic condition of hyperlipidemia. The present invention is also to provide a method of screening an agent for preventing or treating Metabolic Syndrome (especially developed linking to impaired fatty acid utilization, hyperlipidemia, diabetes or the like) by using the non-human mammal model. The present invention is based on new findings of the present inventors in that a non-human mammal whose TBP-2 gene is functionally deficient on chromosome exhibits disorders caused by impaired fatty acid utilization and the almost same pathologic condition as the pathologic condition of hyperlipidemia.

The preferred embodiments of the present invention will be described as bellow. Terms used in the specification includes any meaning which can be interrupted in the art unless otherwise stated. A singular term here include plural concept as well as singular concept unless otherwise stated.

TBP-2 is TRX Binding Protein-2 having the identical molecule with vitamin D-3 induced-protein such as vitamin D3 upregulated protein 1 (VDUP-1) or Thioredoxin interacting protein (Txnip).

A non-human mammal model of disorders caused by impaired fatty acid utilization and a non-human mammal model with pathologic conditions of hyperlipidemia according to the present invention is made by disrupting or impairing functions of TBP-2 gene in animal individuals.

To disrupt or impair functions of TBP-2 genes in mammals, at least the region involved in functions of TBP-2, DNA encoding the TBP-2 or its transcripts is targeted.

This can be done by, for example, preparing mutants, transgenic animal or a knockdown animal. Said mutants includes conditional mutants and are obtained by introducing mutation (deficiency, replacement, addition, insertion) toward TBP-2 gene using homologous recombination. Said transgenic animal has protein mutated protein having dominant-negative mutation. Said knockdown animal is obtained by using antisense RNA or RNAi, or DNA encoding them.

Alternatively, a material which can interfere with or decrease the functions of TBP-2 gene in animal individuals may be used for disrupting or impairing functions of TBP-2 gene.

In the present invention, a non-human mammal whose TBP-2 gene is functionally deficient on chromosome, as mentioned above, is the non-human mammal losing the function to express TBP-2 due to inactivation of endogenous gene encoding TBP-2 in the non-human mammal, wherein the endogenous gene is inactivated by gene mutant such as disruption, deficiency, replacement or the like. The above-mentioned non-human mammals may be Rodentia animals such as rats or mice, but not limited to them.

A wild non-human mammal described here means mammals of the same kind as the above-mentioned non-human mammal whose TBP-2 gene is functionally deficient on chromosome. The wild non-human mammal is preferably a littermate of the non-human mammal.

In addition, as the non-human mammal whose TBP-2 gene is functionally deficient on chromosome, the one born according to Mendel's law is preferably used, because such a mammal is obtained together with its littermate, and the littermate can be used as the wild type. Using the non-human mammal whose TBP-2 gene is functionally deficient on chromosome and its littermate achieves accurate comparative experiments.

Hereinafter, a mouse will be taken up as the non-human mammal to discuss the present invention.

As to methods for making TBP-2 knockout mouse (TBP-2(−/−)), any method is adopted as long as the method can generate the non-human mammal whose TBP-2 gene is functionally deficient on chromosome. One example of the methods is following.

Mouse gene library is amplified by PCR method, and then obtained gene segments undergo screening with probes derived from mouse TBP-2 gene. The screened TBP-2 is subcloned with plasmid vector or the like, and determined by restriction enzyme-mapping and DNA sequencing. Next, all or part of the gene encoding TBP-2 is replaced in pMC1 neogene cassette or the like. Intots 3′ end, genes such as diphtheriatoxin A fragment (DT-A) gene, thymidine kinase (HSV-tk) gene of simple herpes virus or the like are introduced to obtain a targeting vector.

The obtained targeting vector is linearized, and introduced into ES cell by electroporation method or the like, and then it undergoes homologous recombination. Among the resultant, ES cell in which the homologous recombination occurs is selected with antibiotics such as G418, Ganciclovir (GANC) or the like. It is preferably confirmed by Southern blotting or the like whether the selected ES cell is a desired object or not. Clone of the confirmed ES cell is microinjected into blastocyst of mice, and then such blastocyst is returned to a foster parent to make chimeric mice. The obtained chimeric mice are crossed with a wild mouse to obtain heterozygous mice (F1 mice: +/−). Male of the obtained heterozygous mice is crossed with female of it to obtain TBP-2 knockout mice (TBP-2^{-/-}). It is confirmed whether TBP-2 is generated in the obtained TBP-2 knockout mice or not by, for example, northern blotting using isolated RNA from the mice. Alternatively, it is confirmed by identifying expression of TBP-2 in the mice by western blotting.

As mentioned above, generated TBP-2 knockout mice is characterized by that they exhibit impaired fatty acid utilization. It is suggested that TBP-2 is fasting-reactive factor and that it is importantly involved in preventing from hemorrhage under fasting condition. This is because TBP-2 knockout mice exhibits hemorrhage in their various organs under fasting condition.

The TBP-2 knockout mice according to the present invention exhibit pathologic conditions like hyperlipidemia as well as disorders caused by the impaired fatty acid utilization. Therefore the TBP-2 knockout mice are effectively used as mouse model of such disorders or with pathologic conditions. Thus such a non-human mammal is useful when analyzing pathologic condition of Metabolic Syndrome (especially developed linking to impaired fatty acid utilization, hyperlipidemia or the like) and interpreting its pathogenic mechanism. It is also used as experimental material for screening an agent for preventing or treating Metabolic Syndrome.

The term “Metabolic Syndrome” used here means disorders caused or developed by daily life style such as eating habit, insufficient exercise, insufficient relaxation, smoking or drinking as well as by genetic factor or external environmental factor including pathogen, harmful substance, stressor or the like. The Metabolic Syndrome is developed together with hypertension, hyperlipidemia, diabetes, obesity or the like. Phenotypic disorder of the non-human mammal model of disorders according to the present invention exhibits almost the same pathologic conditions as the conditions of Metabolic Syndrome, especially, developed linking to impaired fatty acid utilization, hyperlipidemia or the like.

A particular symptom which the non-human mammal model of disorders exhibits is hemorrhage caused by impaired fatty acid utilization under fasting condition.

According to the after-mentioned examples, it is suggested that the impaired fatty acid utilization in the non-human mammal model of disorders according to the present invention is caused by defect of TCA cycle. This “TCA cycle” is known as circle metabolic pathway in mitochondria, including the following three important functions (1) to (3).

(1) pyruvic acid from glycolysis is converted into acetyl CoA, and it enters the TCA cycle, and the acetyl CoA is converted into two molecular of CO₂ by its oxidation.

(2) Hydrogen is trapped in the form of reduced coenzyme such as 3NADH₂⁺ and FADH₂.

(3) TCA cycle functions as an interface of amino-acid metabolism, urea cycle, gluconeogenesis or the other pathway (cross-point of metabolism).

The non-human mammal model according to the present invention exhibits dysregulation of lipid metabolism, dysregulation of glucose metabolism, coagulation dysfunction or the like, and those pathologic conditions are likely caused by the impaired fatty acid utilization.

Hyperlipidemia is the disease in which lipid in blood such as cholesterol or neutral lipid (triglyceride) is more than normal condition. When at least one of the cholesterol (LDL or HDL), neutral lipid (TG), phosphatide or the like among the lipid in blood is more than normal level, he or she is diagnosed as hyperlipidemia. The hyperlipidemia includes hypercholesterolemia, hyperlipidemia caused by neutral lipid and hyperlipidemia caused by HDL. Lipid becomes excessive in blood because of inheritable character, inappropriate meal and insufficient exercise. This causes atherosclerosis which may be followed by various adult diseases such as ischemic heart disease.

Criterion has been reviewed by referring to epidemiologic search concerning correlative between plasma lipid level and incidence rate of atherosclerotic disease such as ischemic heart disease. For example, on criterion adopted by Japan Atherosclerosis Society, patients, who have more than 220 mg/dl of cholesterol level or have more than 150 mg/dl of neutral lipid level, are diagnosed as hyperlipidemia.

Because TBP-2 knockout mice according the present invention are characterized by that they exhibit impaired fatty acid utilization and hemorrhage in their various organs under fasting condition, they are useful when analyzing pathologic condition of Metabolic Syndrome, which is especially developed linking to impaired fatty acid utilization or hyperlipidemia, and interpreting its pathogenic mechanism. It is also used as experimental material for screening an agent for preventing or treating Metabolic Syndrome.

In addition to the above, TBP-2 knockout mice are used as animal model used for analyzing insulin-secreting-regulatory mechanism on diabetes patients, because they exhibit particular pathologic conditions such as hemorrhage under fasting condition or Hyperinsulinemia caused by impaired fatty acid utilization.

Therefore TBP-2 knockout mice according to the present invention can be used for screening an agent for preventing or treating diabetes.

For the other purpose, the non-human mammal model according to the present invention is used as a non-human mammal model of Reye Syndrome, Reye-like Syndrome, fatty acid oxidation defect, acute fatty liver of pregnancy or the like because of pathologic conditions which it exhibits.

Reye Syndrome means a Syndrome following a certain kind of acute viral infection and causing acute encephalopathy and visceral lipid-infiltration. It induces impaired cranial nerve function, impaired liver function and further Hyperammonemia, which are caused by Metabolic disorder of mitochondria.

Reye-like Syndrome means congenital metabolic Syndrome whose symptom is similar to Reye Syndrome's. Such Reye-like Syndrome includes pre-existing diseases such as ammonia Metabolic abnormality, fatty acid Metabolic abnormality, glucide Metabolic abnormality, organic acid Metabolic abnormality, mitochondria Metabolic abnormality or pyruvic acid Metabolic abnormality.

Fatty acid oxidation defect (FAOD) induces acute encephalopathy which is similar to Reye Syndrome causing dysfunction of liver. When accompanying infectious disease or starvation, low-ketotic hypoglycemia may be induced. Because fatty acid is prevented from being degraded (β-oxidation) on FAOD, lipid (neutral lipid) is accumulated in each organs, and then lipid is denatured. The lipid degeneration in cardiac muscle induces impairment of cardiac muscle or cardiac hypertrophy, and the lipid degeneration in skeletal muscle induces weakness of muscle or muscle tone, rhabdomyolysis or fatty liver.

In addition, the non-human mammal model or disorders according to the present invention is useful for directly determining physiological role of “TBP-2” considered important for regulation of various intravital redox as well as TRX or for basal metabolism-activity.

Hereinafter, the method of screening an agent for preventing or treating Metabolic Syndrome according to the present invention is explained.

The method of screening an agent for preventing or treating Metabolic Syndrome (especially developed linking to impaired fatty acid utilization, hyperlipidemia or the like), Reye Syndrome, Reye-like Syndrome, fatty acid oxidation defect or the like includes, but is not limited to, a method comprising administering a test article to the non-human mammals and evaluating (e.g. checking with eyes) them by referring to their hemorrhage level. The administration of the test articles may be oral administration, intravenous administration or the like.

The non-human mammals are, but are not limited to, animals whose TBP-2 gene is functionally deficient on chromosome and wherein they exhibit hemorrhage under fasting condition. Such animals may be preferably rodents such as mice or rats.

The above-method of screening contributes to developing an agent for preventing or treating Metabolic Syndrome, Reye Syndrome or the like. Further it is useful for diagnosing the above-disorder caused by abnormality of TBP-2 gene.

A method of diagnosing impaired fatty acid utilization caused by abnormality of TBP-2 may include extracting TBP-2 gene from a subject, checking its base sequence and finding out its abnormality by comparing it with normal TBP-2 gene.

### EXAMPLES

The examples of the present inventions will be described in more detail as below. It should be understood, however, that the present invention is not limited by these examples.

### [Materials and Methods]

### 1. Generation of TBP-2 Knockout Mice (TBP-2^{-/-})

Genomic fragments for construction of the TBP-2 targeting vector from a bacterial artificial chromosome clone (7C21, Incyte Genomics. Inc.) were subcloned into pT7Blue or pBluescript and then ligated into pLNTK. The targeting vector was transfected into ES cells (line TT2) by electroporation, and cells were then selected for resistance to G418 and ganciclovir. Southern blotting analysis showed that 8 of 188 clones resistant to antibiotics were correctly targeted. We obtained two lines of chimeric mice with a germ-line transmission derived from independent ES cells. The resulting chimeras were crossed back with ICR and C57B/6 to generate TBP-2 knockout mice (hereinafter referred to as TBP-2^{-/-} mice) (expressed as “^{-/-}” in the figs).

Thus, homozygous knockout mice (TBP-2^{-/-} mice) were generated by crossing heterozygous mice (TBP-2^{+/-} mice) (expressed as “^{+/-}” in the figs).

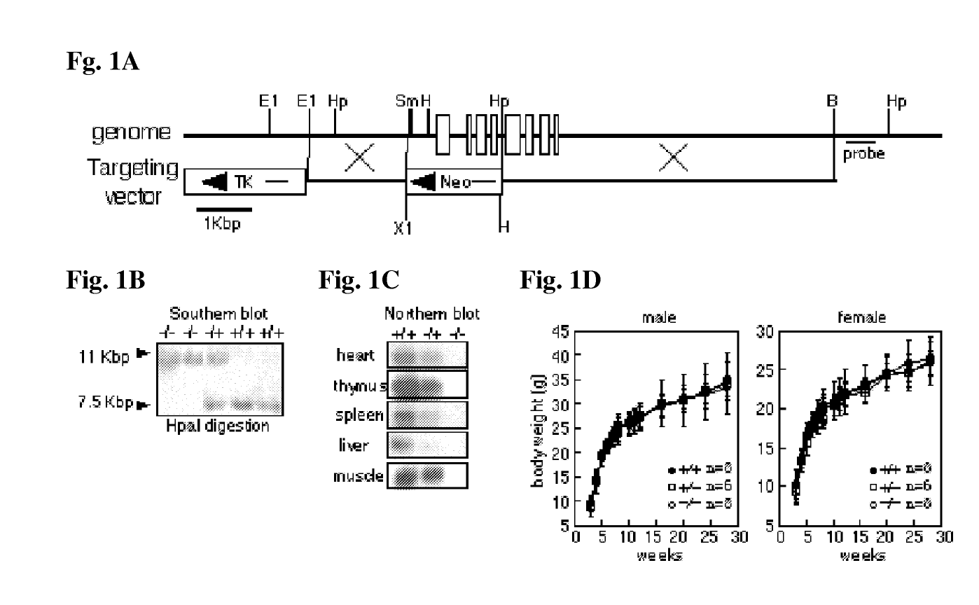

F2 or further backcrossed C57B/6 mice were used. homologous recombination is confirmed by southern blotting analysis using genome DNA which is isolated from tail of the mice as below. Results obtained from ICR-background mice are shown in FIGS. 1B and 1C, while all other data are from C57B/6-background mice.

### 2. Serum Examination and Histopathology

Blood was collected from the tail veins. Triglyceride concentrations were measured using a Wako Triglyceride Test kit according to the manufacturer's instructions (Wako Pure Chemical Industries, Ltd.). Serum insulin levels were quantified using a Mouse Insulin ELISA kit (Shibayagi Co. Ltd.). The other blood assays were performed by Falco Biosystems Ltd. The liver was removed under anesthesia at appropriate time points, and paraffin sections were prepared. Standard hematoxylin-eosin and Oil Red O staining was performed.

### 3. Northern Blot Analysis

Total RNA (for northern blot) was extracted using TRIzol reagent according to the manufacturer's instructions (Invitrogen). Total RNA (10-20 μg/lane) was fractionated by denaturing agarose gel electrophoresis and transferred to a nylon membrane (Hybond N⁺; Amersham Bioscience). The blots were hybridized with a [³²P]-labeled probe prepared using the BcaBest labeling kit (TAKARA).

### 4. Statistical Methods

Results were expressed as the mean standard deviation. Statistical comparisons were made using Student's t test or ANOVA coupled to a Fisher's test. A statistically significant difference was defined as P<0.05. In the data presentation, one asterisk represents P<0.05, two asterisks represent P<0.01, and three asterisks represent P<0.001.

### [Results]

### 1. Generation of TBP-2 Knockout Mice (TBP-2^{-/-})

Disruption of the TBP-2 gene in the TBP-2^{-/-} mice was verified by southern and northern blot analysis (FIGS. 1A, B and C). These mice were viable and fertile, and showed no significant differences in body weight compared to wild type mice (expressed as “^{+/+}” in the figs) (FIG. 1D). No gross appearance of abnormalities was observed up to 18 months of age in the C57B/6 background mice (n=14).

2. Case in which TBP-2 Knockout Mice (TBP-2^{-/-}) Undergo Fasting Condition

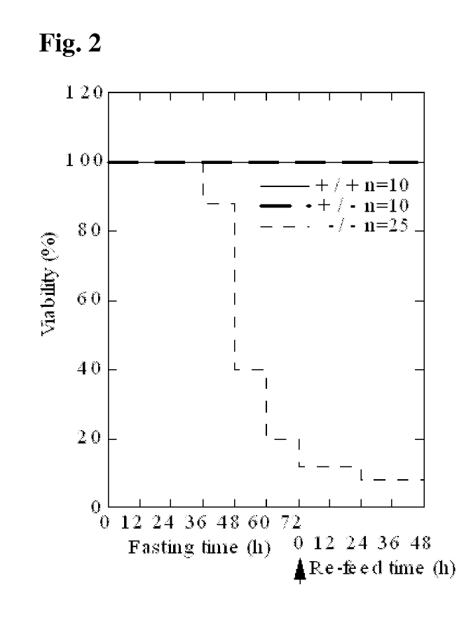

In contrast to their normal appearances under a feeding state, TBP-2^{-/-} mice are predisposed to death under fasting conditions (FIG. 2).

Following 72 or 24 hours fasting, several TBP-2^{-/-} mice could not be rescued by re-feeding, suggesting that irreversible damage had arisen in fasted TBP-2^{-/-} mice.

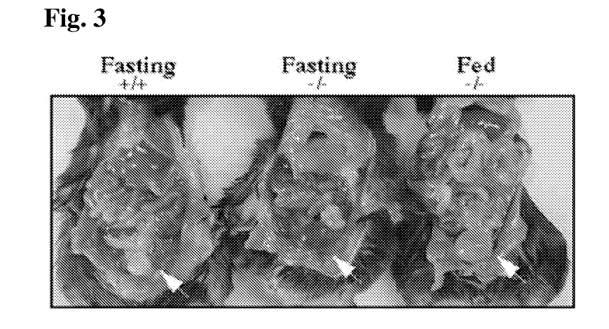

Prior to death, TBP-2^{-/-} mice exhibited coma and hypothermia (data not shown). In addition, hematuria was observed (FIG. 3), which was confirmed by an occult blood test (data not shown).

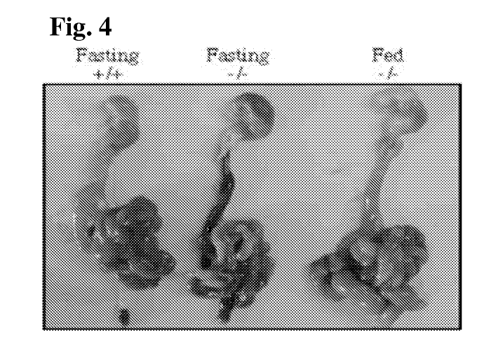

Overall, the gastrointestinal region had a dark red appearance, suggesting that gastrointestinal bleeding was occurring in these mice (FIG. 4). The bleeding tendency was not observed in TBP-2^{-/-} mice during regular feeding.

These results indicate that TBP-2 deficiency leads to a bleeding tendency under fasting condition. The above findings show the importance of TBP-2 in animal survival under fasting conditions, and therefore we investigated the in vivo regulation of TBP-2 expression on fasting. As shown in FIG. 5, TBP-2 expression was significantly up-regulated in the liver, heart and lungs in response to fasting.

These results suggested that TBP-2 is a fasting response gene, and that augmented TBP-2 has an important role in prevention of bleeding under fasting conditions.

### 3. Confirmation of TBP-2^{-/-} Mice-Exhibiting Coagulation Dysregulation Under Fasting Conditions

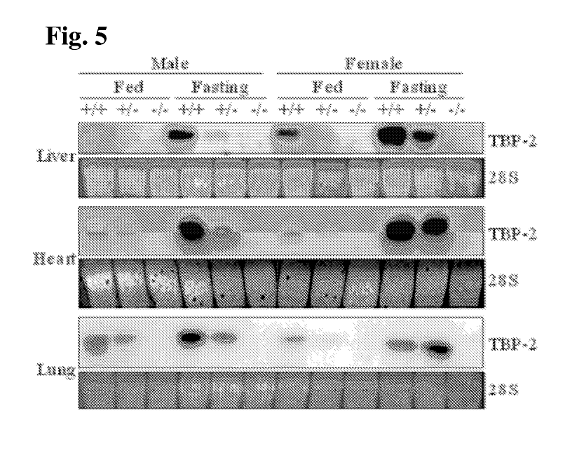

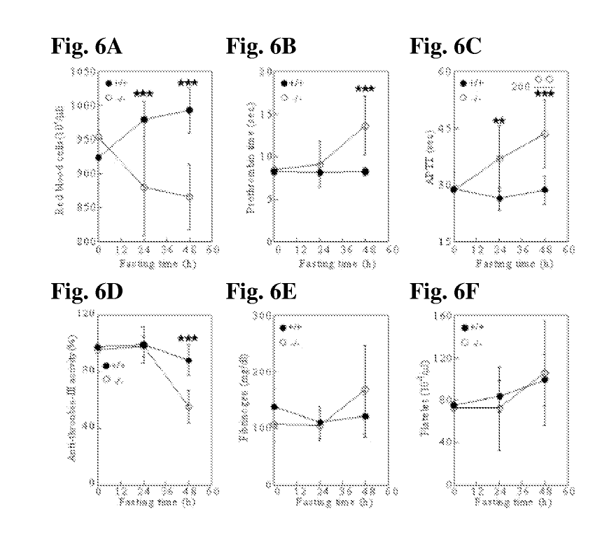

To elucidate the mechanism of hemorrhage in fasted TBP-2^{-/-} mice, serum analyses were performed. A reduction of red blood cells was confirmed in fasted TBP-2^{-/-} mice, which is likely to be the result of hemorrhage (FIG. 6A).

As shown in FIGS. 6B and 6C, prothrombin time (PT) and activated partial thromboplastin time (APTT) did not differ between wild type and TBP-2^{-/-} mice during feeding, whereas they were significantly prolonged in TBP-2^{-/-} mice during fasting. Several mice had a greatly prolonged APTT exceeding 200 seconds, at which time the experiment was stopped. In addition, anti-thrombin III activity was reduced in fasted TBP-2^{-/-} mice compared to wild type mice (FIG. 6D).

These results clearly indicated that TBP-2 deficiency triggers a greatly extended coagulation time during fasting.

Since the above findings lead us to hypothesize that the severe bleeding occurring in TBP-2^{-/-} mice is due to disseminated intravascular coagulation (DIC), which is caused by depletion of fibrinolytic factors resulting from accelerated disseminated coagulation.

DIC is caused by consumption of coagulation factors resulting from hypercoaguability and hyperfibrinolysis, and decreased platelets and fibrinogen are diagnostic of DIC. To investigate whether fasted TBP-2^{-/-} mice have similar characteristics of DIC, plasma levels of fibrinogen and platelet count were examined.

However, neither platelet counts nor plasma fibrinogen levels were significantly changed in TBP-2^{-/-} mice, compared to wild type mice (FIGS. 6E and 6F).

Thus, the hemorrhage did not result from DIC in TBP-2^{-/-} mice.

### 4. Liver Steatosis and Multi-Organ Dysfunction Occurring in Fasted TBP-2^{-/-} Mice

The liver in TBP-2^{-/-} mice was yellow in color during fasting (FIG. 3), indicating a macroscopic liver abnormality.

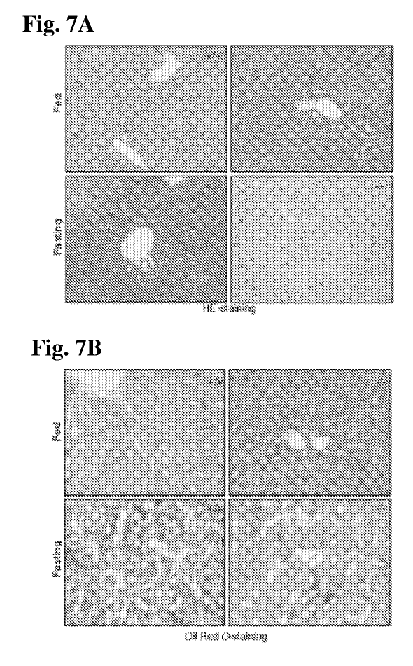

Accordingly, histological studies were performed and, as shown in FIG. 7A, HE staining showed microvesicular and macrovesicular steatosis in TBP-2^{-/-} mice after 24 hours fasting. The fatty liver was also confirmed by staining using Oil Red O (FIG. 7B). Liver steatosis continued for 48 hours fasting until just before death (data not shown).

Liver steatosis is a cause of liver failure and, in addition, severe bleeding results in multi-organ failure, including failure of the liver and kidney.

To examine whether such organ failure may be associated with the hemorrhage in fasted TBP-2^{-/-} mice, serum analyses were performed.

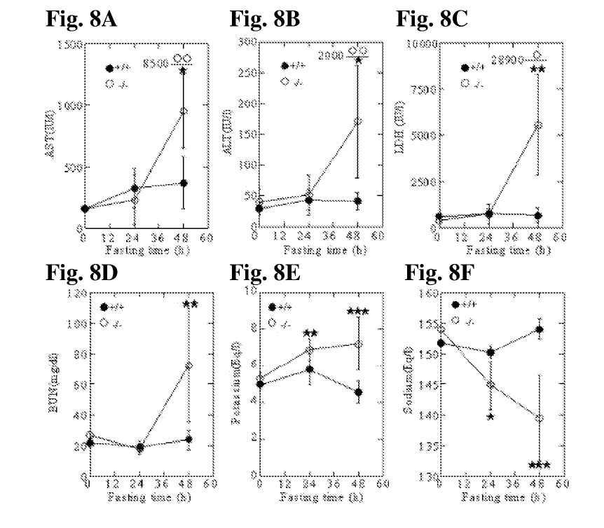

The levels of alanine aminotransferase (ALT), aspartate aminotransferase (AST) and lactate dehydrogenase (LDH) were elevated in TBP-2^{-/-} mice during fasting, compared to wild type mice (FIGS. 8C, 8D and 8E), indicating that liver damage is induced in TBP-2^{-/-} mice during fasting.

The levels of blood urea nitrogen (BUN) also increased in TBP-2^{-/-} mice compared to wild type mice during fasting (FIG. 8F), and TBP-2^{-/-} mice exhibited both hyponatremia and hyperkalemia during fasting (FIGS. 8G and 8H).

Thus, renal failure was induced in TBP-2^{-/-} mice during fasting.

These results suggested that hemorrhage as well as multi-organ failure occurs in fasted TBP-2^{-/-} mice.

### 5. Impaired Fatty Acid Utilization in TBP-2^{-/-} Mice

As mentioned above, it has been concluded that glucose supplementation corrected the fatal anomaly in TBP-2^{-/-} mice, while fatty acids were unable to do so. This will be described in more detail as below by the following test examples.

Glucose and fatty acids are known to be the major energy sources from foods. The anomaly observed in TBP-2^{-/-} mice strongly indicates the occurrence of nutritional dysmetabolism. To examine this hypothesis, these mice were housed with free access to 20% glucose or 10% oleic acid in drinking water, while deprived of other food.

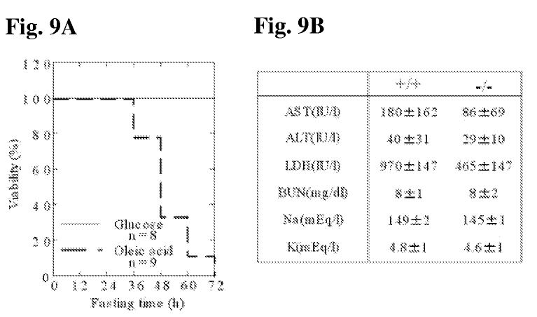

With 20% glucose available in drinking water, TBP-2^{-/-} mice survived for 3 days, whereas TBP-2^{-/-} mice were not rescued with 10% oleic acid (FIG. 9A). Wild type mice were able to survive under both conditions (data not shown).

Serum analyses revealed that glucose supplementation completely blocked the dysfunction of hepatocyte and renal cells in TBP-2^{-/-} mice (FIG. 9B), and hemorrhage and fatty liver were also prevented by glucose supplementation (data not shown).

In contrast, TBP-2^{-/-} mice with oleic acid supplementation showed identical symptoms to fasted TBP-2^{-/-} mice (data not shown), suggesting that fatty acid utilization is functionally impaired in TBP-2^{-/-} mice.

Since fatty acid is a major energy source under fasting conditions, these results suggested that the fatal phenotype is triggered by glucose-deprivation, due to impaired fatty acid utilization in TBP-2^{-/-} mice.

To investigate glucose mobilization, serum glucose levels were measured.

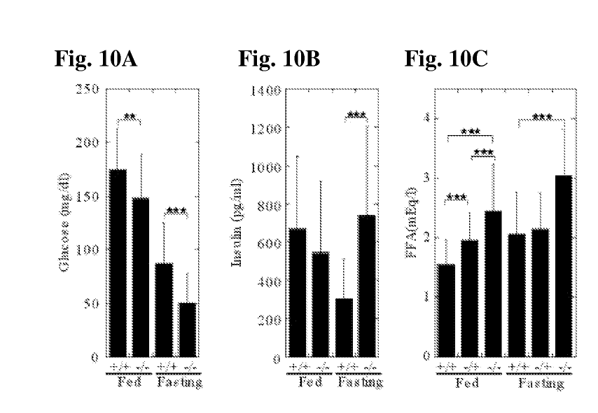

The levels of glucose were decreased in TBP-2^{-/-} mice under both feeding and fasting states (FIG. 10C), suggesting that TBP-2^{-/-} mice preferentially utilize glucose as an energy source. Since insulin is a hormone that promotes glucose utilization, serum insulin levels were examined. Insulin levels were not significantly changed on feeding, whereas fasting-induced reduction of insulin was not observed in TBP-2^{-/-} mice (FIG. 10D). Thus, paradoxical hyperinsulinemia is induced in TBP-2^{-/-} mice.

Several reports have shown that impaired fatty acid utilization enhances insulin secretion, although the precise mechanism remains to be elucidated. To investigate fatty acid mobilization, the levels of free fatty acids (FFA) were examined. As shown in FIG. 10E, serum FFA levels were elevated in both TBP-2^{+/-} and TBP-2^{-/-} mice in a feeding state, and increased in TBP-2^{-/-} mice under fasting conditions. Thus, deposition of FFA is sharply correlated with TBP-2 deficiency.

Since insulin promotes lipogenesis and inhibits lipid consumption, it was thought that hyperinsulinemia and impaired fatty acid utilization may cooperatively lead to dyslipidemia under fasting conditions.

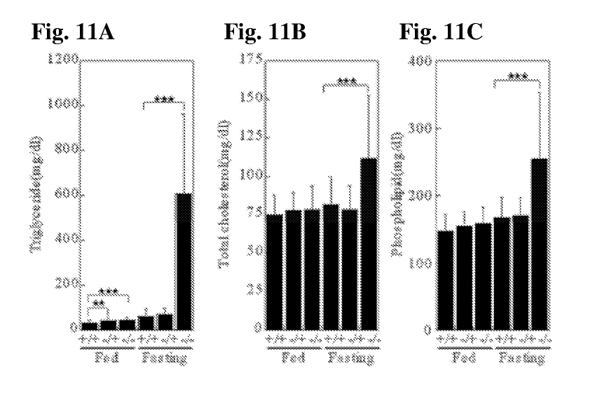

Therefore, the serum levels of lipid compounds were measured. The levels of serum triglyceride were slightly higher in both TBP-2^{+/-} and TBP-2^{-/-} mice in a feeding state, and significantly elevated in fasted TBP-2^{-/-} mice (FIG. 11F). In addition, the levels of total cholesterol and phospholipids were increased in TBP-2^{-/-} mice during fasting (FIGS. 11G and 11H).

Thus, dyslipidemia arises in TBP-2^{-/-} mice under fasting conditions, rather than in a feeding state.

Taken together, these observations suggested that deterioration of fatty acid utilization is a primary change that occurs with TBP-2 deficiency.

### 6. Acetyl-CoA Catabolism is Dysregulated in TBP-2^{-/-} Mice

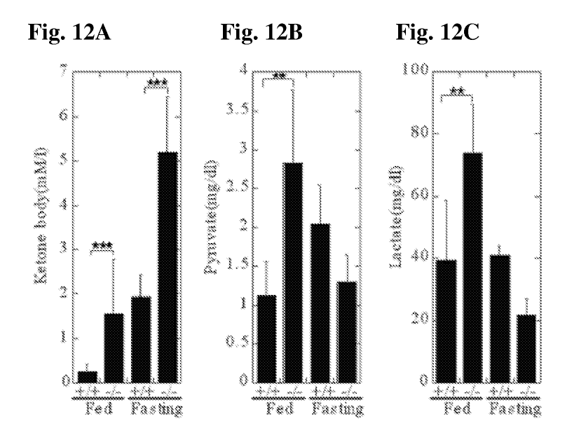

The mechanism underlying the impaired fatty acid utilization in TBP-2^{-/-} mice was examined. Fatty acids are converted into acetyl-CoA through β-oxidation, and the acetyl-CoA is then consumed by the Krebs cycle. Alternatively, excess amounts of acetyl-CoA may be converted into ketone bodies. To determine whether β-oxidation is defective in TBP-2^{-/-} mice, the serum levels of ketone bodies were examined. Ketone bodies were found to accumulate in TBP-2^{-/-} mice during feeding, compared with wild type mice, and were further elevated on fasting (FIG. 12A). These results suggest that β-oxidation is unimpaired, but that acetyl-CoA consumption is reduced in TBP-2^{-/-} mice. Acetyl-CoA is also derived from glucose via pyruvate, and excess amounts of pyruvate are converted into lactate.

Hence, to further investigate whether acetyl-CoA utilization is reduced in TBP-2^{-/-} mice, the levels of pyruvate and lactate were examined in TBP-2^{-/-} mice. As shown in FIGS. 12B and 12C, both pyruvate and lactate were elevated in TBP-2^{-/-} mice during feeding. Although these metabolites decreased in fasted TBP-2^{-/-} mice, this is almost certainly due to the reduction of glucose levels.

Overall, these results suggested that Krebs cycle-mediated acetyl-CoA consumption is reduced in TBP-2^{-/-} mice.

### [Discussion]

Under normal housing conditions, TBP-2^{-/-} mice are viable and fertile, but under fasting conditions, their survival rate was sharply reduced, concomitant with severe bleeding, dyslipidemia, fatty liver, hypoglycemia, and hepatic and renal dysfunction.

These results suggest that these fatal abnormalities are caused by impaired fatty acid utilization in TBP-2^{-/-} mice.

These anomalies are similar to fatty acid utilization deficient disorders, and therefore the TBP-2^{-/-} mouse might be a novel animal model of disorders such as Reye Syndrome, and particularly of AFLP, since this is associated with severe bleeding. The complication of AFLP occurs when the fetus has a homozygous defect of LCHAD (Long-Chain 3 Hydroxyacyl CoA Dehydrogense), which is an important enzyme for β-oxidation, but the precise mechanism through which the β-oxidation defect leads to coagulation disorder remains to be elucidated. Although the specific responsible gene is apparently different, hemorrhage may be induced by a common mechanism in TBP-2^{-/-} mice and LCHAD deficiency.

Peroxisome proliferator-activated receptor-α (PPAR-α is activated by fatty acid ligands and accelerates fatty acid utilization through transcriptional activation of several enzymes promoting β-oxidation. PPAR-α also regulates the transcription of coagulation-regulating genes such as fibrinogen and plasminogen activator inhibitor. Since fatty acid utilization is defective in PPAR-α null mice, these mice show phenotypes that are quite similar to those of TBP-2^{-/-} mice, such as high susceptibility to fasting-induced death, hyperinsulinemia and dyslipidemia. However, bleeding tendency has not been reported in PPAR-α null mice. We have several pieces of evidence which suggest that the activity of PPAR-α is enhanced in TBP-2^{-/-} mice, compared to wild type mice (data not shown), which is certainly due to accumulation of fatty acids (FIG. 10E). Aberrant PPAR-α activation may be a cause of coagulation dysregulation in TBP-2^{-/-} and LCHAD deficiency. Further investigation of the bleeding mechanism in fasted TBP-2^{-/-} mice might elucidate the link between energy metabolism and blood coagulation.

Hyperinsulinemia, hypoglycemia, dyslipidemia and fatty liver have also been reported under fasting conditions in the spontaneous hyperlipidemia mouse strain, HcB-19. The authors concluded that hyperinsulinemia is the primary alteration, which in turn leads to hypoglycemia and hyperlipidemic phenotypes in HcB-19 mice. Our present data strongly suggest that the N-terminal truncated form of TBP-2 in HcB-19 mice causes a loss of function, since quite similar results were obtained in TBP-2 null mice. Furthermore, we suggest that reduced acetyl-CoA consumption is the more primary defect, and this is the cause of hyperinsulinemia in TBP-2^{-/-} mice during fasting. HcB-19 mice show reduced CO₂ production as well as expression of several enzymes regulating the Krebs cycle and the electron transport chain. These observations from TBP-2^{-/-} and HcB-19 mice strongly suggest insufficient functioning of the Krebs cycle and/or the electron transport chain in TBP-2-deficient mice. However, the precise molecular mechanism is currently unclear, and further investigation needs to be performed.

Since TBP-2 inhibits the reducing activity of TRX, it is possible that some aspects of the phenotype of TBP-2^{-/-} mice may be explained by loss of TBP-2-dependent up-regulation of TRX activity. However, under either fed or fasting states, the expression levels of TRX were not significantly altered in the liver of TBP-2^{-/-} mice, compared to wild type mice (data not shown). These data are also consistent with those from HcB-19 mice. Furthermore, TRX-overexpressing transgenic mice did not exhibit dyslipidemia (data not shown). Taken together, an increased level of free TRX per se does not seem to be directly involved in the TBP-2^{-/-} mouse phenotype.

The present inventors have found that TBP-2 binds to TRX-2, a mitochondria-specific protein of the TRX family, suggesting that TBP-2 is a common binding partner for the TRX protein family (Masutani and Wang et al., unpublished data). Several mitochondrial proteins include a conserved consensus sequence of the TRX redox active site (Cys-X-X-Cys), including CPT and cytochrome c. TBP-2 is localized in the mitochondria and binds to cytochrome c in vivo, and therefore TBP-2 may elicit its action through interaction with a mitochondrial TRX-like protein. It has also been suggested that TBP-2 is involved in tumor suppression. Numerous studies have revealed that carcinogenesis is associated with reorganization or dysregulation of basic energy metabolism, including augmentation of the glycolytic pathway and reduction of Krebs cycle function. Since TBP-2^{-/-} mice displayed reduced Krebs cycle activity and augmentation of the glycolytic pathway, carcinogenesis-linked Metabolic changes appear to be consistent with down-regulation of TBP-2 expression.

Hence, TBP-2 may exert a tumor-suppressor activity through maintenance of an anti-carcinogenic Metabolic phenotype.

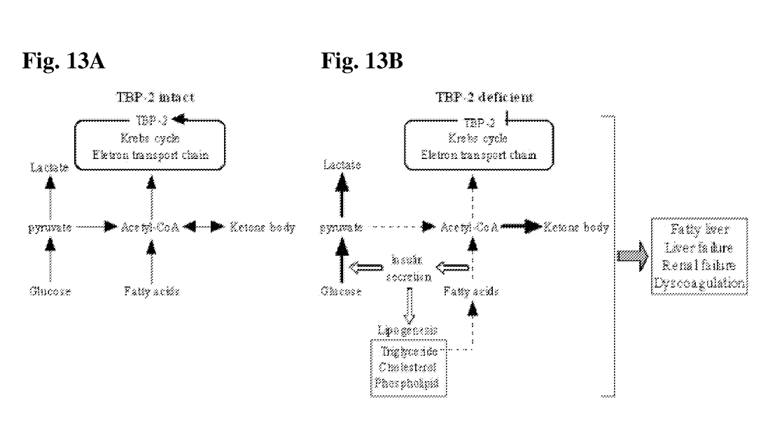

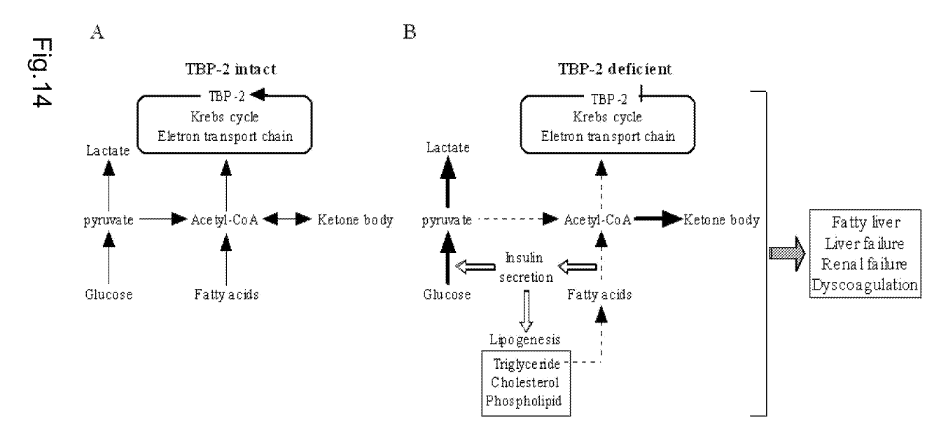

In conclusion, TBP-2 plays an important role in fatty acid utilization through augmentation of Krebs cycle-mediated acetyl-CoA utilization (FIG. 13A).

Disruption of TBP-2 reduces acetyl-CoA consumption, which serially leads to dysregulation of lipid and glucose metabolism, hepatocyte and renal failure, and coagulation dysfunction (FIG. 13B).

Although it remains unclear whether TBP-2 is involved in human diseases such as Reye Syndrome, our present study provides some intriguing implications and possibilities for the role of TBP-2 in these disorders.

Hence, the TBP-2^{-/-} mouse may represent an animal model that could be a useful tool for the study of such disorders and the evaluation of therapeutic approaches for use.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A method of screening an agent for preventing or treating at least one metabolic syndrome, disorder or pathologic condition comprising the step of:
a) administering a test article to a non-human mammal comprising a TBP-2 gene, wherein the gene is functionally deficient on a chromosome.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The method of claim 18, wherein the metabolic syndrome, disorder or pathologic condition is a disorder caused by impaired fatty acid utilization.

35. The method of claim 34, further comprising the steps of:
b) administering the test article to a wild non-human mammal;
c) comparing conditions between the two non-human mammals; and
d) evaluating the conditions.

36. The method of claim 18, wherein the metabolic syndrome, disorder or pathologic condition is a metabolic syndrome.

37. The method of claim 36, further comprising the steps of:
b) administering the test article to a wild non-human mammal;
c) comparing conditions between the two non-human mammals; and
d) evaluating the conditions.

38. The method of claim 18, wherein the metabolic syndrome, disorder or pathologic condition is hyperlipidemia.

39. The method of claim 38, further comprising the steps of:
b) administering the test article to a wild non-human mammal;
c) comparing conditions between the two non-human mammals; and
d) evaluating the conditions.

40. The method of claim 18, wherein the metabolic syndrome, disorder or pathologic condition is diabetes.

41. The method of claim 40, further comprising the steps of:
b) administering the test article to a wild non-human mammal;
c) comparing conditions between the two non-human mammals; and
d) evaluating the conditions.

42. The method of claim 18, wherein the metabolic syndrome, disorder or pathologic condition is selected from a group consisting of Reye Syndrome, Reye-like Syndrome, fatty acid oxidation defect and acute fatty liver of pregnancy.

43. The method of claim 42, further comprising the steps of:
b) administering the test article to a wild non-human mammal;
c) comparing conditions between the two non-human mammals; and
d) evaluating the conditions.

44. The method of claim 18, wherein the metabolic syndrome, disorder or pathologic condition is a pathologic condition selected from a group consisting of dysregulation of lipid metabolism, dysregulation of glucose metabolism and coagulation dysfunction.

45. The method of claim 44, further comprising the steps of:
b) administering the test article to a wild non-human mammal;
c) comparing conditions between the two non-human mammals; and
d) evaluating the conditions.

46. The method of claim 18, wherein the non-human mammal is a rodent.

47. The method of claim 46, further comprising the steps of:
b) administering the test article to a wild non-human mammal;
c) comparing conditions between the two non-human mammals; and
d) evaluating the conditions.

47. The method of claim 18, wherein the non-human mammal is a mouse.

48. The method of claim 47, further comprising the steps of:
b) administering the test article to a wild non-human mammal;
c) comparing conditions between the two non-human mammals; and
d) evaluating the conditions.

49. The method of claim 18, further comprising the steps of:
b) administering the test article to a wild non-human mammal;
c) comparing conditions between the two non-human mammals; and
d) evaluating the conditions.
